(19) United States
(12) Patent Application Publication
(10) Pub. No.: US 2020/0397901 A1
(43) Pub. Date: Dec. 24, 2020

(54) PHOTORESPONSIVE SMOOTHENED LIGAND

(71) Applicant: The University of Tokyo, Tokyo (JP)

(72) Inventors: Yoshiho Ikeuchi, Tokyo (JP); Ryuji Misawa, Tokyo (JP)

(73) Assignee: The University of Tokyo, Tokyo (JP)

(21) Appl. No.: 16/957,806

(22) PCT Filed: Jan. 24, 2019
(86) PCT No.: PCT/JP2019/002189

§ 371 Date: Jul. 23, 2020

(30) Foreign Application Priority Data
Jan. 24, 2018 (JP) 2018-009258

(51) Int. Cl. A61K 41/00; C07D 409/12; C12N 13/00

(52) U.S. Cl. CPC A61K 41/0042; C12N 13/00; C07D 409/12

(57) ABSTRACT

The present invention is intended to provide a photoresponsive Smo ligand, in which a photodissociable protective group binds to an active site of a Smo ligand (a site important for agonistic or antagonistic activity). Specifically, the present invention relates to a photoresponsive Smo ligand, in which a photodissociable protective group binds to an active site of a Smo ligand. More specifically, it is a photoresponsive Smo ligand, which is characterized in that the Smo ligand is a 1,4-diaminocyclohexane derivative and the photodissociable protective group comprises an aromatic ring.

### TECHNICAL FIELD

The present invention relates to a Smoothened ligand that is activated in response to light.

### BACKGROUND ART

Hedgehog signaling pathway is a signaling pathway having a hedgehog protein as a center, wherein the hedgehog signaling pathway plays an important role for individual cells to obtain correct location information in the development of an embryo or regeneration of tissues. Inhibition of this pathway may cause severe developmental disorder, etc., and may lead to lethal events in some cases (Non Patent Literature 1). It has been reported that abnormal hedgehog signals are associated with the formation and progression of tumors, such as osteoblastoma, basal cell carcinoma and rhabdomyosarcoma, in adult bodies (Non Patent Literature 2, Non Patent Literature 3, etc.). Thus, studies in which the hedgehog pathway is treated as a target of drug discovery have been promoted. For example, agonists of the hedgehog pathway have attracted attention to be utilized as therapeutic agents for cerebral ischemia such as cerebral infarction or cerebral hemorrhage (Non Patent Literature 4). On the other hand, antagonists of the hedgehog pathway have attracted attention to be utilized as anticancer agents (Non Patent Literature 2, Non Patent Literature 3, etc.).

Hedgehog signaling is initiated by a hedgehog protein. In a case where such a hedgehog protein is not present, a 12-transmembrane receptor, Patched (Ptch), suppresses the activity of Smoothened (Smo) that is a seven-transmembrane GPCR-like receptor. If Smo is inactivated in vertebrates, Sufu (Suppressor of Fused) binds to the transcriptional factor Gli to form a heterodimer, and phosphorylation of a Gli protein is promoted, so as to generate GliR in which the N-terminus is cleaved. The thus generated GliR functions as a repressor of a hedgehog response gene, and suppresses transcription of hedgehog target genes such as GLI1 or PTCH1.

On the other hand, if the hedgehog protein binds to Ptch, suppression of Smo is released, and the cleavage of the N-terminal side of the Gli protein is suppressed, so as to generate full-length Gli (Gli^{A}) that is an active type. This active type Gli transfers to the nucleus, and induces transcription of hedgehog target genes (Non Patent Literature 4).

For the purpose of treating developmental disorder, tumorigenesis and the like caused by abnormality in the hedgehog pathway, agonists or antagonists of the hedgehog pathway have been developed.

It has been demonstrated that various cancers, such as osteoblastoma, rhabdomyosarcoma, brain tumor, basal cell carcinoma, lung cancer and breast cancer, are caused by excessive activation of the hedgehog pathway. Hence, the use of the hedgehog pathway antagonists as candidates of anticancer agents has been expected, and a large number of antagonists have been reported (Patent Literature 1, Non Patent Literature 3, Non Patent Literature 5, and Non Patent Literature 6).

Meanwhile, agonists of the hedgehog pathway have been expected as therapeutic agents for developmental disorder, neurodegenerative disease, bone degenerative disease, diabetes, and obesity, as well as ischemia such as ischemic heart disease or cerebral ischemic disease (Non Patent Literature 4). At present, SAG (Smoothened agonist) (Non Patent Literature 4, Non Patent Literature 5, and Non Patent Literature 6), Pumorphamine (Non Patent Literature 4), and the like, which directly bind to Smo, have been known as well-studied agonists. SAG is a small molecule compound that binds to a heptahelical domain of Smo, and several compounds having a common skeleton have been reported. Even regarding substituents important for agonistic activity, studies have been conducted intensively (Non Patent Literature 4).

With regard to the hedgehog protein that plays a main role in the hedgehog pathway, three types of hedgehog homologs, namely, Sonic hedgehog, Indian hedgehog, and Desert hedgehog haven been identified in mammals. Among these, Sonic hedgehog has been most studied. Sonic hedgehog functions as a morphogen, for example, in determination of the axis of a neural tube in the early development of the brain. In recent years, attempts have been made to culture pluripotent stem cells as an aggregate, and to induce differentiation of three-dimensional tissues. Many attempts have been vigorously made to utilize organoids, such as artificial brain tissues (Non Patent Literature 7, Non Patent Literature 8, etc.) or lung epithelium (Non Patent Literature 9), as human disease models.

However, artificial brain tissues obtained in homogeneous culture media, which have been used in the existing reports, are not able to determine the axis by themselves, and therefore, various brain sites are formed disorderly. Under such circumstances, the artificial brain tissues are far from being practiced as a disease model.

### CITATION LIST

### Patent Literature

Patent Literature 1: US2010/0048637 A1

Patent Literature 2: US6,683,108 B1

### Non Patent Literature

Non Patent Literature 1: Zhang et al., Cell 106: 781-792, 2001

Non Patent Literature 2: Sekulic and Von Hoff, Cell 164: 831, 2016

Non Patent Literature 3: Rimkus et al., Cancers 8, 22: 2016 Doi: 10.3390/cancers8020022

Non Patent Literature 4: Hadden, ChemMedChem 9: 27-37, 2014

Non Patent Literature 5: Yang et al., J. Biol. Chem. 284: 20876-20884, 2009

Non Patent Literature 6: King, J. Biol. 1(2), 8, 2002

Non Patent Literature 7: Lancaster et al., Nature 501: 373-379, 2013

Non Patent Literature 8: Di Lullo et al., Nature Review neuroscience 18: 573-584, 2017

Non Patent Literature 9: Dye et al., eLife 4, Doi:10.7554/eLife.05098, 2015

### SUMMARY OF INVENTION

### Technical Problem

Under the aforementioned circumstances, it is an object of the present invention to provide a photoresponsive Smoothened agonist (photoresponsive Smo agonist) utilized in induction of artificial tissues, in which constituent cells are properly arranged, and in the treatment of developmental disorder, neurodegenerative disease, bone degenerative disease, diabetes, obesity, ischemia such as ischemic heart disease or cerebral ischemic disease, etc., and a photoresponsive Smoothened antagonist (photoresponsive Smo antagonist) that can be utilized in the treatment of cancer and the like caused by abnormality in the hedgehog pathway (hereinafter, the agonist and the antagonist are collectively referred to as a “photoresponsive Smo ligand”).

### Solution to Problem

Activation of the Smo receptor in the right place at the right time in embryonic development is extremely important for body axis formation. On the other hand, in a case where cancer cells are suppressed by inactivation of the Smo receptor, if the inactivation of the Smo receptor affected even normal cells, side effects would be provoked.

In view of the foregoing, the present inventors have attempted to develop a photoresponsive Smo ligand, by which only the Smo ligand existing in a desired position in tissues (including artificial tissues) is activated. The inventors have selected SAG as a representative Smo ligand, and have bound a photoresponsive protective group to an amino group thereof that is important for the activity of SAG. As a result, the SAG activity disappeared. Subsequently, the present inventors have applied a light to the photoresponsive protective group-binding SAG (hereinafter also referred to as “caged SAG”), and as a result, they have confirmed that the SAG activity is recovered.

The present invention relates to a “photoresponsive Smo ligand,” which has been completed based on the aforementioned findings.

Specifically, the present invention relates to a photoresponsive Smo ligand, in which a photodissociable protective group binds to an active site of a Smo ligand (a site important for agonistic or antagonistic activity).

More specifically, it is the above-described photoresponsive Smo ligand, which is characterized in that the Smo ligand is SAG or a SAG derivative (including an antagonist) and the photodissociable protective group comprises an aromatic ring.

Moreover, the present invention relates to a method of inducing artificial tissues in vitro, comprising culturing cells or tissues in the presence of the above-described photoresponsive Smo ligand.

Furthermore, the present invention relates to a medicament or a pharmaceutical composition, comprising, as an active ingredient, the above-described photoresponsive Smo ligand, a salt thereof, or a solvate or a hydrate thereof.

### Advantageous Effects of Invention

According to the present invention, it becomes possible to control hedgehog signals at a high spatio-temporal resolution, and thus, a means for elucidating various functions of the hedgehog pathway is provided.

Further, according to the present invention, it becomes possible to develop a therapeutic agent and a therapeutic method for diseases caused by abnormal hedgehog signaling.

### BRIEF DESCRIPTION OF DRAWINGS

FIG. 1 shows the results obtained by evaluating the properties of caged SAG according to absorption spectrum measurement. The upper view is an absorption spectrum graph of caged SAG and SAG. The lower view is a graph showing differences in absorption spectral changes of caged SAG subjected to light irradiation with different energy intensities.

FIG. 2 shows charts obtained by analyzing caged SAG irradiated with a light in PBS according to HPLC (detected by absorption at 270 nm), and the results (within the frames) obtained by analyzing molecules eluted around 23 minutes and fractions eluted around 14 minutes according to ESI Mass.

FIG. 3 shows graphs obtained by plotting the peak areas of the caged SAG and the SAG in the HPLC charts shown in FIG. 2 to the amount of the irradiated light.

FIG. 4 shows induction of the expression of ventral cerebrum markers in cerebral organoids using caged SAG after completion of light irradiation. The upper view is a view showing the outline of the experiment. The lower view shows the results obtained by measuring the expression levels of the ventral cerebrum markers (NKX2.1 and MASH1) in cerebral organoids that have been cultured in the presence of SAG, caged SAG before light irradiation, and caged SAG after completion of light irradiation.

FIG. 5 shows the results obtained by immunohistostaining cerebral organoids that have been cultured in the presence of caged SAG after completion of light irradiation, with an anti-NKX2.1 antibody and an anti-MASH1 antibody.

FIG. 6 shows the results obtained by applying a light to NIH-3T3 cells cultured in the presence of caged SAG, and then examining the expression level of a hedgehog signal downstream gene in the cells. The upper view is a view showing the outline of the experiment. The lower view shows the results obtained by applying a light to NIH-3T3 cells cultured in the presence of caged SAG, and then examining the expression level of Gli in the cells.

FIG. 7 shows the results obtained by analyzing the relationship between the amount of SAG and the expression level of Gli. The upper view is a view showing the outline of the experiment. The lower view shows the results obtained by measuring the expression level of Gli in NIH-3T3 cells cultured in the presence of SAG and caged SAG (with or without light irradiation).

FIG. 8 shows the results obtained by applying a light to cerebral organoids cultured in the presence of caged SAG, and then examining the expression levels of ventral cerebrum markers. The upper view is a view showing the outline of the experiment. The lower view shows the results obtained by applying a light to cerebral organoids cultured in the presence of caged SAG, and then measuring the expression levels of the ventral cerebrum markers (NKX2.1 and MASH1).

FIG. 9 shows the results obtained by analyzing the properties of IR-783-SANT75 (SANT75: a SAG derivative having antagonistic activity; and IR-783: a photodissociable protective group).

FIG. 10 shows the results obtained by confirming the recovery of antagonistic activity by applying a light to NVOC-SANT75 (NVOC: a photodissociable protective group).

### DESCRIPTION OF EMBODIMENTS

A first embodiment of the present invention relates to a photoresponsive Smo ligand, in which a photodissociable protective group binds to an active site of a Smo ligand.

The term “Smo ligand” is used herein to mean a substance that binds to a seven-transmembrane GPCR-like receptor, Smoothened, and controls the activity thereof, positively (Smo agonist) or negatively (Smo antagonist). To date, there have been many study reports regarding the Smo ligand (see, for example, Patent Literature 1, Patent Literature 2, Non Patent Literature 3, Non Patent Literature 4, Non Patent Literature 5, and Non Patent Literature 6). More specifically, examples of the Smo ligand may include, but are not particularly limited to, 1,4-diaminocyclohexane derivative, which is referred to as SAG and a SAG derivative, and pumorphamine and a derivative thereof (see, for example, US2010/0048637 A1, Hadden, ChemMedChem 9: 27-37, 2014, Yang et al., J. Biol. Chem. 284: 20876-20884, 2009, King, J. Biol. 1(2), 8, 2002, Brunton et al., Bioorg Med Chem Lett. 19: 4308-4311, 2009, Seifer et al., Bioorg Med Chem. 20: 6465-6481, 2012, and Che et al., Beilstein J Org Chem. 8: 841-849, 2012).

In the photoresponsive Smo ligand according to the first embodiment (hereinafter also referred to as “the photoresponsive Smo ligand of the present invention”), a photodissociable protective group binds to an active site of a known Smo ligand (i.e., a site important for agonistic or antagonistic activity). The photoresponsive Smo ligand of the present invention is characterized in that it exhibits almost no activity (neither agonistic nor antagonistic activity) in the aforementioned state, but when a light is applied to the photoresponsive Smo ligand of the present invention so that the photodissociable protective group is dissociated, the present photoresponsive Smo ligand exhibits its original activity.

The active site of the Smo ligand can be specified by methods well known in the present technical field, such as structure-activity relationships (SAR). In the case of 1,4-diaminocyclohexane derivative (SAG and a derivative thereof) that is one example of the Smo ligand, the active site is present in the portion of an amino group binding to cyclohexane (i.e., an amino group to which a benzothiophene ring, a furan ring, or the like does not bind). For example, in the 1,4-diaminocyclohexane derivative shown in the following formula (I), if H that binds to N to which R₁ binds is substituted with a bulky functional group, such as a benzyl group or an n-butyl group containing an aromatic ring, its activity is lost (see Non Patent Literature 5, etc.). Thus, this portion is considered to be an active site.

In the first embodiment of the present invention, the term “photodissociable protective group” is used to mean any given functional group that is dissociated by light irradiation, and the photodissociable protective group preferably comprises a bulky structure such as an aromatic ring (see, for example, Klan et al., Chem. Rev. 113: 119-191, 2013). Such a photodissociable protective group can be easily selected by a person skilled in the art, and examples of the photodissociable protective group may include, but are not particularly limited to, a group having a 2-nitrobenzyl derivative skeleton, a dimethoxybenzoin group, a 2-nitropiperonyloxy carbonyl (NPOC) group, a 2-nitroveratryloxycarbonyl (NVOC) group, an α-methyl-2-nitropiperonyloxycarbonyl (MeNPOC) group, an α-methyl-2-nitroveratryloxycarbonyl (MeNVOC) group, a 2,6-dinitrobenzyloxycarbonyl (DNBOC) group, an α-methyl-2,6-dinitrobenzyloxycarbonyl (MeDNBOC) group, a 1-(2-nitrophenyl)ethyloxycarbonyl (NPEOC) group, a 1-methyl-1-(2-nitrophenyl)ethyloxycarbonyl (MeNPEOC) group, a 9-anthracenylmethyloxycarbonyl (ANMOC) group, a 1-pyrenylmethyloxycarbonyl (PYMOC) group, a 3′-methoxybenzoinyloxycarbonyl (MBOC) group, a 3′,5′-dimethoxybenzoyloxycarbonyl (DMBOC) group, a 7-nitroindolinyloxycarbonyl (NIOC) group, a 5,7-dinitroindolinyloxycarbonyl (DNIOC) group, a 2-anthraquinonylmethyloxy carbonyl (AQMOC) group, an α,α-dimethyl-3,5-dimethoxybenzyloxycarbonyl group, and a 5-bromo-7-nitroindolinyloxy carbonyl (BNIOC) group.

Further, when the photoresponsive ligand of the present invention is used as a medicament, the light to be applied to the present photoresponsive ligand to dissociate the photodissociable protective group therefrom needs to have a wavelength that causes low ecotoxicity and high tissue permeability. Accordingly, it is desirable to use a photodissociable protective group that is dissociated from the present photoresponsive ligand by being irradiated with, for example, near infrared light (wavelength: approximately 650 nm to 900 nm). Examples of such a photodissociable protective group may include C4′-dialkylamine-substituted heptamethine cyanines (see Groka et al., J. Am. Chem. Soc., 136: 14153-14159, 2014; Nani et al., Angew. Chem. Int. Ed., 54:13635-13638 2015; etc.). When a photoresponsive ligand, to which a photodissociable protective group that is dissociated by being irradiated with such near infrared light binds, is administered to a living body and then reaches a desired site, it can be activated as a ligand at the desired site by being irradiated with a light (near infrared light), and thus, the photoresponsive ligand can be used in the treatment of disease.

Examples of a preferred Smo ligand used in the first embodiment of the present invention may include, but are not particularly limited to, SAG and SAG derivatives disclosed in, for example, US2010/0048637 A1, Hadden, ChemMedChem 9: 27-37, 2014, Yang et al., J. Biol. Chem. 284: 20876-20884, 2009, King, J. Biol. 1(2), 8, 2002, Brunton et al., Bioorg Med Chem Lett. 19: 4308-4311, 2009, Seifer et al., Bioorg Med Chem. 20: 6465-6481, 2012, and Che et al., Beilstein J Org Chem. 8: 841-849, 2012. Herein, the SAG derivatives include not only Smo agonists but also Smo antagonists. As specifically described later, there has been known a case where the SAG derivative functions as an antagonist, depending on the type of a substituent represented by R₁ in the following formula (I).

Herein, the SAG and the SAG derivative may be, for example, but are not particularly limited to, a 1,4-diaminocyclohexane derivative represented by the following formula (I).

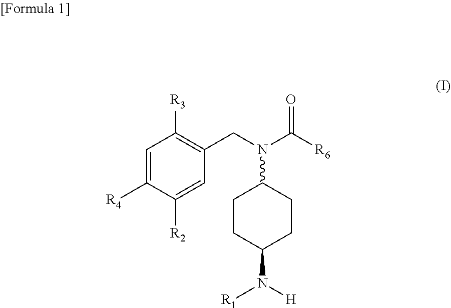

In the above formula (I), R₁ is a hydrogen atom, or a saturated or unsaturated hydrocarbon group having 1 to 6 carbon atoms, and is preferably a hydrogen atom, a methyl group, an ethyl group, a propyl group, or a 2-propenyl group. In particular, when the Smo ligand of the first embodiment is an agonist, R₁ is preferably hydrogen or a methyl group (see Non Patent Literature 4 and Non Patent Literature 5). When the Smo ligand of the first embodiment is an antagonist, R₁ is preferably an ethyl group, a propyl group, or a 2-propenyl group (see Non Patent Literature 5).

R₂ is hydrogen, a substituted or unsubstituted phenyl group, or a substituted or unsubstituted pyridyl group, and is particularly preferably a phenyl group, a 4-cyanophenyl group, a 4-methoxyphenyl group, a 4-(methylsulfonyl)phenyl group, a 3-cyanophenyl group, or a 4-pyridinyl group.

R₃ is hydrogen, fluorine, or a hydrocarbon group having 1 to 3 carbon atoms and optionally containing oxygen, and is particularly preferably hydrogen, fluorine, a methyl group, or a methoxy group.

R₄ is hydrogen, a methoxy group, or a substituent represented by the following formula (1), and R₅ is preferably a hydrocarbon chain having 1 to 4 carbon atoms and optionally containing an amide bond.

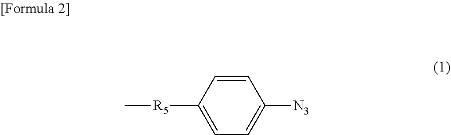

R₆ is a substituent containing a substituted or unsubstituted aromatic ring (including a heteroaromatic ring), and is preferably any substituent represented by the following formula (2), (3), (4) or (5). In the formula (5), R₇ is preferably chlorine or a methyl group, and R₈ is preferably hydrogen or fluorine.

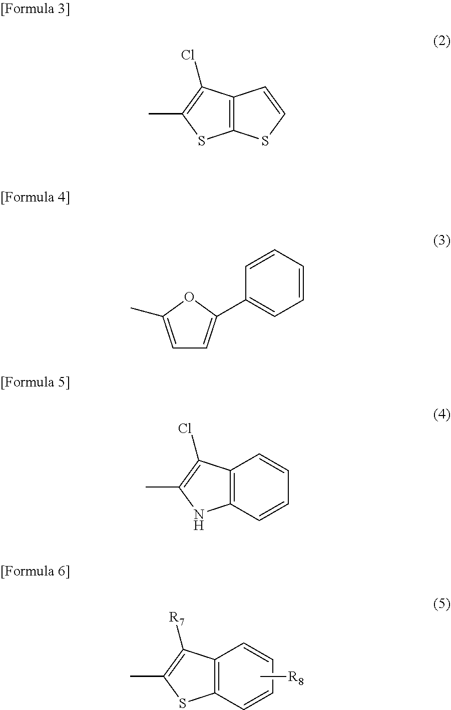

The SAG or the SAG derivative represented by the formula (I) can be synthesized with reference to, for example, Non Patent Literature 5, Frank-Kamenetsky et al., J. Biol. 1(2), 8, 2002, Wnag et al., J. Comb. Chem. 10: 825-834, 2008, James et al., Proc Natl Acad Sci USA 99: 14071-14076, 2002, Che et al., Beilstein J Org Chem. 8: 841-849, 2012, etc. Otherwise, a commercially available product may be purchased.

The photoresponsive Smo ligand of the present invention is not particularly limited, and it may be, for example, a photoresponsive Smo ligand, in which a photodissociable protective group comprising an aromatic ring binds to the active site of the aforementioned SAG or SAG derivative. As one example, the photoresponsive Smo ligand of the present invention can be represented, for example, by the following formula (II), but is not particularly limited thereto.

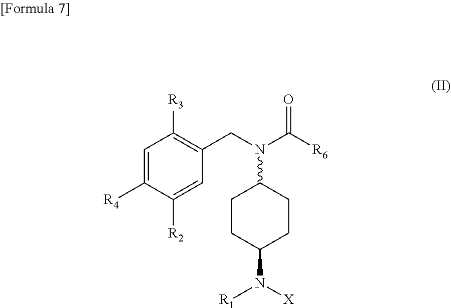

In the above formula (II), X is a photodissociable protective group.

R₁ is a hydrogen atom, or a saturated or unsaturated hydrocarbon group having 1 to 6 carbon atoms, and is preferably a hydrogen atom, a methyl group, an ethyl group, a propyl group, or a 2-propenyl group. In particular, when the Smo ligand of the first embodiment is an agonist, R₁ is preferably hydrogen or a methyl group (see Non Patent Literature 4 and Non Patent Literature 5). When the Smo ligand of the first embodiment is an antagonist, R₁ is preferably an ethyl group, a propyl group, or a 2-propenyl group (see Non Patent Literature 5).

R₂ is hydrogen, a substituted or unsubstituted phenyl group, or a substituted or unsubstituted pyridyl group, and is particularly preferably a phenyl group, a 4-cyanophenyl group, a 4-methoxyphenyl group, a 4-(methylsulfonyl)phenyl group, a 3-cyanophenyl group, or a 4-pyridinyl group.

R₃ is hydrogen, fluorine, or a hydrocarbon group having 1 to 3 carbon atoms and optionally containing oxygen, and is particularly preferably hydrogen, fluorine, a methyl group, or a methoxy group.

R₄ is hydrogen, a methoxy group, or a substituent represented by the following formula (1), and R₅ is preferably a hydrocarbon chain having 1 to 4 carbon atoms and optionally containing an amide bond.

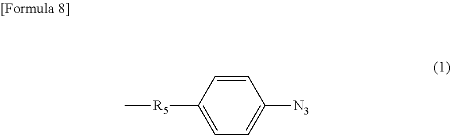

R₆ is a substituent containing a substituted or unsubstituted aromatic ring (including a heteroaromatic ring), and is preferably any substituent represented by the following formula (2), (3), (4) or (5). In the formula (5), R₇ is preferably chlorine or a methyl group, and R₈ is preferably hydrogen or fluorine.

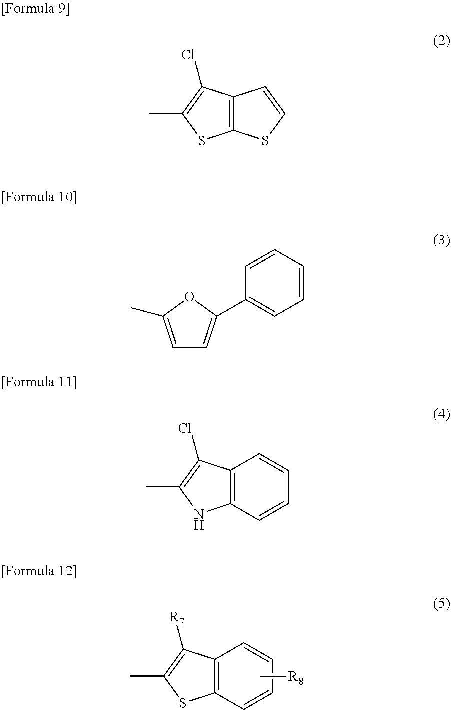

Examples of the photoresponsive ligand of the present invention, which is an agonist, may include the compound groups shown in Table 1 to Table 5 below. R₁ to R₈ in Table 1 to Table 4 indicate the substituents shown in formula (II′) below, and R₁ to R₈ in Table 5 indicate the substituents shown in formula (II″).

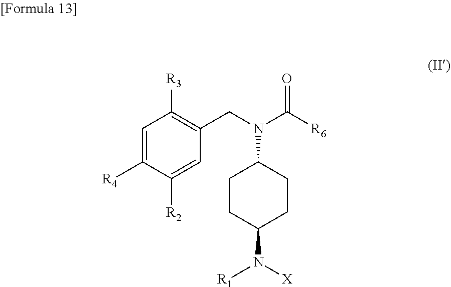

TABLE 1

| R₁ | R₂ | R₃ | R₄ | R₅ | R₆ | R₇ | R₈ |
|---|---|---|---|---|---|---|---|
| Methyl group | 4-Pyridinyl group | H | H | | 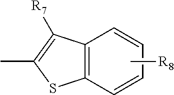 | Cl | H |
| Methyl group | 4-Pyridinyl group | H | H | | 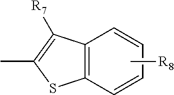 | C1 | 4,7-F |
| Methyl group | 4-Pyridinyl group | Methoxy group | H | | 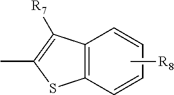 | Cl | 4,7-F |
| H | 4- Cyanophenyl group | H | Methoxy group | | 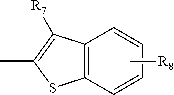 | Cl | H |
| Methyl group | 4- Cyanophenyl group | H | Methoxy group | | 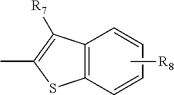 | Cl | H |
| Methyl group | 4- Cyanophenyl group | H | Methoxy group | | 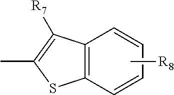 | Cl | 4,7-F |
| Methyl group | H | H | 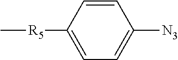 | 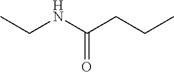 | 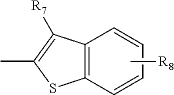 | Cl | H |

See Hadden, ChemMedChem 9: 27-37, 2014; King, J. Boil. Volume 1, Issue 2, Article 8, 2002

TABLE 2

| R₁ | R₂ | R₃ | R₄ | R₆ | R₇ | R₈ |
|---|---|---|---|---|---|---|
| Methyl group | 4-Pyridinyl group | H | H | 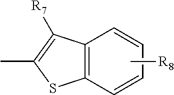 | Cl | 4-Cl |
| Methyl group | 4-Pyridinyl group | H | H | 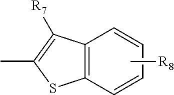 | Cl | 6-F |
| Methyl group | 4-Pyridinyl group | H | H | 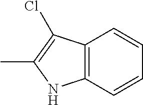 | | |
| Methyl group | 4-Pyridinyl group | H | H | 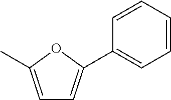 | | |

See Seifer et al., Bioorg Med Chem, 20: 6465-6481, 2012

TABLE 3

| R₁ | R₂ | R₃ | R₄ | R₆ | R₇ | R₈ |
|---|---|---|---|---|---|---|
| Methyl group | 4-Cyanophenyl group | H | Methoxy group | 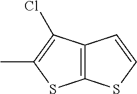 | | |
| Methyl group | 4-Cyanophenyl group | H | Methoxy group | 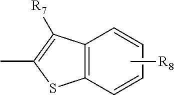 | Cl | 4-F |
| Methyl group | 4-Cyanophenyl group | H | Methoxy group | 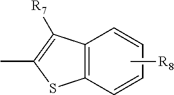 | Cl | 5-F |
| Methyl group | 4-Cyanophenyl group | H | Methoxy group | 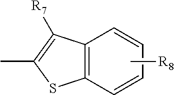 | Cl | 7-F |
| Methyl group | 4-Cyanophenyl group | H | Methoxy group | 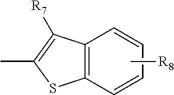 | Cl | 6-F |
| H | 4-Cyanophenyl group | H | Methoxy group | 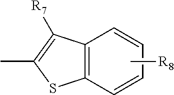 | Methyl group | H |

See Brunton et al., Bioorg Med Chem Lett. 19: 4308-4311, 2009

TABLE 4

| R₁ | R₂ | R₃ | R₄ | R₆ | R₇ | R₈ |
|---|---|---|---|---|---|---|
| Methyl group | 4-Cyanophenyl group | Methoxy group | H | 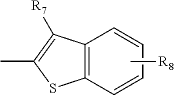 | C1 | H |
| Methyl group | Phenyl group | Methoxy group | H | 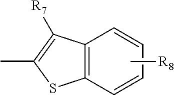 | Cl | H |
| Methyl group | 3-Cyanophenyl group | Methoxy group | H | 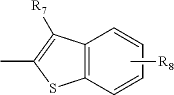 | Cl | H |
| Methyl group | 3-(Methylsulfonyl)phenyl group | Methoxy group | H | 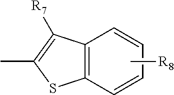 | Cl | H |
| Methyl group | 4-Methoxyphenyl group | Methoxy group | H | 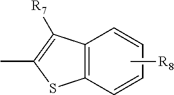 | Cl | H |
| Methyl group | 4-Cyanophenyl group | F | H | 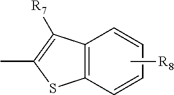 | Cl | H |
| Methyl group | 4-Cyanophenyl gorup | H | H | 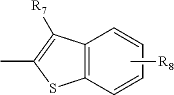 | Cl | H |
| Methyl group | 4-Cyanophenyl group | Methyl group | H | 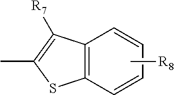 | Cl | H |
| Methyl group | 4-Pyridinyl group | H | Methoxy group | 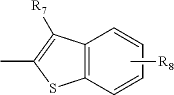 | Cl | H |
| Methyl group | 4-Cyanophenyl group | H | Methoxy group | 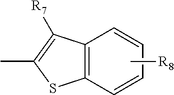 | Cl | 4-F |

See Brunton et al., Bioorg Med Chem Lett. 19: 4308-4311, 2009

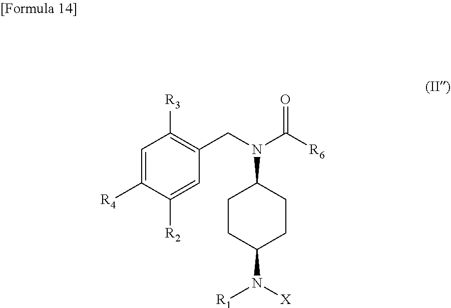

TABLE 5

| R₁ | R₂ | R₃ | R₄ | R₆ | R₇ | R₈ |
|---|---|---|---|---|---|---|
| Methyl group | 4-Pyridinyl group | H | H | 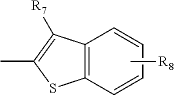 | Cl | 4-Cl |
| Methyl group | 4-Pyridinyl group | H | H | 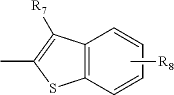 | Cl | H |

See Seifer et al., Bioorg Med Chem, 20: 6465-6481, 2012

Moreover, the photoresponsive ligand of the present invention, which is an antagonist, may be the following compound group.

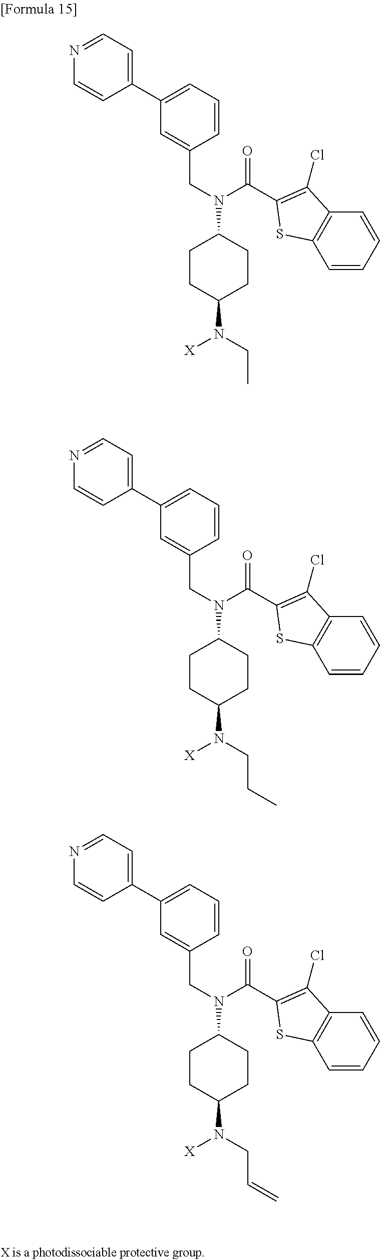

A second embodiment of the present invention relates to a medicament or a pharmaceutical composition, comprising, as an active ingredient, the photoresponsive Smo ligand of the present invention, a salt thereof, or a solvate or a hydrate thereof.

The photoresponsive ligand of the present invention can be used in the treatment of disease caused by abnormal hedgehog signaling. The photoresponsive Smo agonist can be used in the treatment of, for example, developmental disorder, neurodegenerative disease, bone degenerative disease, diabetes, obesity, and ischemia such as ischemic heart disease or cerebral ischemic disease (Hadden, ChemMedChem 9: 27-37, 2014). On the other hand, the photoresponsive Smo antagonist can be used in the treatment of cancers such as osteoblastoma, rhabdomyosarcoma, brain tumor, basal cell carcinoma, lung cancer, and breast cancer (Sekulic and Von Hoff, Cell 164: 831, 2016; Rimkus et al., Cancers 8, 22: 2016, Doi: 10.3390/cancers 8020022).

With regard to the medicament according to the second embodiment of the present invention, the photoresponsive Smo ligand of the present invention, or a salt thereof, or a solvate or a hydrate thereof may directly be administered. However, in general, the medicament may be desirably administered in the form of a pharmaceutical composition comprising such a substance serving as an active ingredient, and one or two or more pharmaceutical additives.

In addition, two or more types of photoresponsive Smo ligands of the present invention may be used in combination, as active ingredients of the medicament according to the embodiment of the present invention. The above-described pharmaceutical composition may further comprise known other components effective for the treatment of the target disease.

Examples of the dosage form of the medicament or the pharmaceutical composition according to the present invention may include a tablet, a capsule, a granule, a powder agent, a syrup agent, a suspending agent, a suppository, an ointment, a cream agent, a gelling agent, a patch, an inhalant, and an injection. These pharmaceutical agents are prepared according to common methods. Besides, in the case of a liquid preparation, it may be dissolved or suspended in water or other suitable solvents when it is used. Further, in the case of a tablet or a granule, it may be coated according to a publicly known method. In the case of an injection, it is prepared by dissolving the compound of the present invention in water. The compound may also be dissolved in a normal saline or a glucose solution, as necessary, or a buffer or a preservative may be added to the injection.

A preparation for use in oral administration or parenteral administration is provided in any given preparation form. Examples of the preparation form that can be prepared herein may include: medicaments or pharmaceutical compositions for use in oral administration, having the form of a granule, a fine granule, a powder agent, a hard capsule, a soft capsule, a syrup agent, an emulsion, a suspending agent, a liquid agent, etc.; and medicaments or pharmaceutical compositions for use in parenteral administration, having the form of an injection for intravenous administration, intramuscular administration or subcutaneous administration, drops, a transdermal agent, a transmucosal agent, nasal drops, an inhalant, a suppository, etc. In the case of an injection or drops, a powdery dosage form such as a freeze-dried form may be prepared, and when it is used, it may be dissolved in an appropriate aqueous medium such as a normal saline and may be then used.

The type of the pharmaceutical additive used in the production of the medicament or the pharmaceutical composition according to the present invention, the proportion of the pharmaceutical additives to the active ingredient, or a method for producing the medicament or the pharmaceutical composition can be appropriately selected by a person skilled in the art, depending on the form thereof. As such a pharmaceutical additive, an inorganic or organic substance, or a solid or liquid substance can be used. In general, the pharmaceutical additive can be used in an amount of 1% by weight to 90% by weight, with respect to the weight of the active ingredient. Specifically, examples of the pharmaceutical additive may include lactose, glucose, mannite, dextrin, cyclodextrin, starch, sucrose, magnesium aluminometasilicate, synthetic aluminum silicate, sodium carboxymethyl cellulose, hydroxypropyl starch, calcium carboxymethyl cellulose, an ion exchange resin, methyl cellulose, gelatin, gum Arabic, hydroxypropyl cellulose, hydroxypropyl methyl cellulose, polyvinyl pyrrolidone, polyvinyl alcohol, light anhydrous silicic acid, magnesium stearate, talc, tragacanth, bentonite, veegum, titanium oxide, sorbitan fatty acid ester, sodium lauryl sulfate, glycerin, fatty acid glycerin ester, purified lanolin, glycerogelatin, polysorbate, macrogol, vegetable oil, wax, liquid paraffin, white petrolatum, fluorocarbon, a nonionic surfactant, propylene glycol, and water.

In order to produce a solid preparation for oral administration, the active ingredient was mixed with an excipient component such as, for example, lactose, starch, crystalline cellulose, calcium lactate or anhydrous silicic acid to form a powder agent, or further, as necessary, a binder such as white sugar, hydroxypropyl cellulose or polyvinyl pyrrolidone, a disintegrator such as carboxymethyl cellulose or calcium carboxymethyl cellulose, or other agents are added to the powder agent, and the obtained mixture is then subjected to wet or dry granulation to form a granule. In order to produce a tablet, such a powder agent or a granule may be tableted, directly or after addition of a lubricant such as magnesium stearate or talc. Such a granule or a tablet may be coated with an enteric coated agent base such as hydroxypropylmethyl cellulose phthalate or a methacrylic acid-methyl methacrylate polymer to prepare an enteric coated preparation, or may be coated with ethyl cellulose, carnauba wax, hydrogenated oil or the like to prepare a sustained-release preparation. In addition, in order to produce a capsule, such a powder agent or a granule may be filled into a hard capsule, or the active ingredient may be coated with a gelatin film, directly or after it has been dissolved in glycerin, polyethylene glycol, sesame oil, olive oil or the like, so as to prepare a soft capsule.

In order to produce an injection, the active ingredient may be dissolved in distilled water for injection, as necessary, together with a pH adjuster such as hydrochloric acid, sodium hydroxide, lactose, lactic acid, sodium, sodium monohydrogen phosphate or sodium dihydrogen phosphate, and a tonicity agent such as sodium chloride or glucose, and thereafter, the obtained mixed solution may be subjected to aseptic filtration and may be then filled into an ampoule. Otherwise, mannitol, dextrin, cyclodextrin, gelatin or the like may be further added to the reaction mixture, and the obtained mixture may be then freeze-dried under vacuum to prepare a use-time dissolution type injection. Otherwise, lecithin, polysorbate 80, polyoxyethylene hydrogenated castor oil or the like may be added to the active ingredient, and the obtained mixture may be then emulsified in water to prepare an emulsion for injection.

In order to produce a rectal administration agent, the active ingredient is humidified and dissolved together with a suppository base such as cacao butter, tri-, di- and mono-glyceride of fatty acids, or polyethylene glycol, and is then poured into a mold, followed by cooling. Otherwise, the active ingredient may be dissolved in polyethylene glycol, soybean oil or the like, and may be then coated with a gelatin film.

The medicament or the pharmaceutical composition according to the present invention is not particularly limited in terms of administration amount and administration frequency, and the administration amount and the administration frequency can be selected, as appropriate, by doctor's judgement, depending on the purpose of prevention and/or treatment of deterioration and/or progression of a therapeutic target disease, the type of the disease, conditions such as the body weight and age of a patient.

In general, the daily dose of the medicament or the pharmaceutical composition according to the present invention administered to an adult by oral administration is approximately 0.01 to 1000 mg (the weight of the active ingredient), and the medicament or the pharmaceutical composition can be administered once or divided over several administrations per day, or every several days. In the case of using the present medicament or pharmaceutical composition as an injection, it is desirably administered, continuously or intermittently, to an adult at a daily dose of 0.001 to 100 mg (the weight of the active ingredient).

The medicament or the pharmaceutical composition according to the present invention can be prepared into a sustained-release preparation such as an implant tablet or a delivery system encapsulated into a microcapsule, using a carrier capable of preventing the prompt removal of the medicament or the pharmaceutical composition from the inside of a body. Examples of such a carrier that can be used herein may include biodegradable biocompatible polymers such as ethylene vinyl acetate, polyanhydride, polyglycolic acid, collagen, polyorthoester, and polylactic acid. Such a material can be easily prepared by a person skilled in the art. In addition, a liposome suspension can also be used as a pharmaceutically acceptable carrier. Although the type of such a liposome is not limited, it can be prepared as a lipid composition comprising phosphatidylcholine, cholesterol and PEG-derivatized phosphatidylethanol (PEG-PE) and having a size suitable for the use, by being passed through a filter having a suitable pore size, and it can be then purified according to a reverse phase evaporation method.

The medicament or the pharmaceutical composition according to the present invention may be provided in the form of a kit together with instruction manuals regarding administration method, etc. The drug included in the kit is supplied by using a container, which effectively sustains the activity of the constituent components of the medicament or pharmaceutical composition for a long period of time, is not adsorbed on the inner side of the container, and is produced from a material that does not deteriorate the constituent components. For example, a sealing glass ampoule may contain a buffer that has been enclosed in the presence of a neutral unreactive gas such as nitrogen gas

Moreover, use instructions may be included with the kit. The use instructions of the kit may be printed on a paper and the like, or may be stored in an electromagnetically readable medium such as CD-ROM or DVD-ROM, so that the use instructions may be supplied to a user.

Moreover, a third embodiment of the present invention relates to a method of administering the medicament or the pharmaceutical composition according to the second embodiment of the present invention to a therapeutic target, so as to treat developmental disorder, neurodegenerative disease, bone degenerative disease, diabetes, obesity, ischemia (ischemic heart disease, cerebral ischemic disease, etc.), or cancer (e.g., osteoblastoma, rhabdomyosarcoma, brain tumor, basal cell carcinoma, lung cancer, breast cancer, etc.).

Herein, the term “treatment” means to prevent or alleviate the progression and deterioration of the pathologic condition of a mammalian animal affected with a disease or the like, so as to treat the disease for the purpose of preventing or alleviating the progression or deterioration of the disease.

The “mammalian animal” as a target of the treatment means any given animal classified into mammals. Examples of the mammalian animal may include, but are not particularly limited to, humans, companion animals such as a dog, a cat and a rabbit, and livestock animals such as a bovine, a swine, sheep and a horse. The “mammalian animal” is particularly preferably a human.

A fourth embodiment of the present invention relates to a method of inducing artificial tissues in vitro, comprising:

a step of culturing cells or tissues in the presence of the photoresponsive Smo ligand of the present invention, and a step of irradiating the cultured cells or tissues with a light.

Herein, the term “artificial tissue” means a culture having a function and/or a structure similar to those of any given biological tissues, which is obtained by culturing pluripotent stem cells in vitro. More specifically, it is a culture having a function and/or a structure similar to biological tissues, such as, for example, brain, lung, or intestine. Further, in the present description, the artificial tissue has a concept including an organ in which different tissues are combined with one another.

In the photoresponsive Smo ligand of the present invention, a photodissociable protective group that binds to the ligand is irradiated with a light having a wavelength suitable for degradation of the photodissociable protective group, so that the protective group is dissociated and the ligand is thereby activated. Cells or tissues are cultured in the presence of the photoresponsive Smo ligand of the present invention, and the cultured cells or tissues are then irradiated with a light in the right place at the right time, and the Smo ligand is thereby activated, so that cells or a cell population having desired characteristics can be induced to differentiate in a desired tissue location. For instance, pluripotent stem cells (iPS cells, etc.) are cultured to become a spheroid (cell mass), and a light is then applied to a desired site on the spheroid in the presence of the photoresponsive Smo ligand of the present invention, which serves as an agonist, so that the Smo agonist can be activated at the site, a hedgehog pathway can be activated, and desired cell differentiation can be induced. In contrast, when the activated hedgehog signals are to be suppressed, a light is applied to a desired site on the tissues in the presence of the photoresponsive Smo ligand of the present invention, which serves as an antagonist, so that the hedgehog signals can be suppressed at the desired site. As a result, induced cell differentiation can be suppressed at the desired site.

As described above, by applying a light to a desired location on a cell mass cultured in the presence of the photoresponsive Smo ligand of the present invention (an agonist and/or an antagonist), it becomes possible to induce tissues having a desired three-dimensional structure.

With regard to the wavelength and energy intensity of a light irradiated, it is desirable to adopt a wavelength suitable for the photodissociable protective group that binds to the used ligand, and then to apply a light at an intensity that does not affect the cells and is sufficient for the dissociation of the protective group. The wavelength of the light to be irradiated can be easily selected by a person skilled in the art. Otherwise, the energy intensity of the light to be irradiated can be determined according to preliminary experiments.

The disclosures of all publications cited in the present description are incorporated herein by reference in their entirety. In addition, throughout the present description, when singular terms such as “a,” “an,” and “the” are used, these terms include not only single items but also multiple items, unless otherwise clearly specified.

Hereinafter, the present invention will be further described in the following examples. However, these examples are only provided as examples of the embodiments of the present invention, and are not intended to limit the scope of the present invention.

### EXAMPLES

### 1. Experimental Methods

### 1-1. Culture of Human iPS Cells

The cell line iPSC 409B2 (Cell no. HPS0076) was obtained from National University Corporation Kyoto University through RIKEN BioResource Center Cell Bank. In order to maintain the undifferentiated state, the iPS cells were cultured in an Essential 8 medium on a dish coated with vitronectin (VNT-N, Thermofisher Scientific).

### 1-2. Culture of NIH-3T3 Cells and Hedgehog Signal Assay

NIH-3T3 cells were allowed to grow in DMEM (supplemented with 10% FBS, glutamine, penicillin, and streptomycin). In order to perform a hedgehog signal assay, the cells that had been cultured to reach confluent were cultured in a starvation-induced medium (DMEM; serum not added) overnight, so that the cells were in a starved state. Subsequently, the medium was exchanged with a starvation-induced medium supplemented with SAG, caged SAG, SANT75, caged SANT75, or the like. Using a halogen lamp, the cells were irradiated with a light for 15 minutes. Thereafter, total RNA was extracted from the NIH-3T3 cells cultured for 6 hours, and was then subjected to real-time PCR.

### 1-3. Preparation of Cerebral Organoid

A cerebral organoid was prepared according to the method described in Lancaster et al., Nat Protoc. 9: 2329-2340, 2014. Using TrypLE™ (Thermofisher Scientific), hiPSCs cultured in an E8 medium were separated into single cells, which were then reaggregated to a cell mass having a density of approximately 10,000 cells, using a 100 4/well E6 medium, in a low-adhesion 96-well round bottom plate.

To the E6 medium, FGF2 (5 ng/mL, 0 to 4 days after initiation of the culture) and a ROCK inhibitor (Thiazovivine, 1μM, 0 to 4 days after initiation of the culture) were added. The medium was exchanged with a fresh one at 2 and 4 days after initiation of the culture. At 6 days after initiation of the culture, the medium was exchanged with a neural differentiation medium (DMEM/F12, 1% N2 supplement, insulin, 1% GlutaMax supplement, 1% NEAA, 1% penicillin-streptomycin, and 1 μg/mL heparin) containing SAG, caged SAG (photoresponsive protective group-binding SAG), or caged SAG before or after UV irradiation. When caged SAG was added, the culture was irradiated with a light for 15 minutes, using a halogen lamp. At 6, 8, and 10 days after initiation of the culture, the medium was exchanged with a fresh one.

Three types of organoids cultured under individual conditions were recovered at 11 days after initiation of the culture, and were then subjected to qPCR. Each organoid was embedded in a Matrigel droplet (25 μl), and was then transferred on a 60-mm dish containing a brain differentiation medium (DMEM/F12 : Neurobasal™ media=1:1, which was supplemented with a 0.5% N2 supplement, a 1% GlutaMax supplement, 0.5% NEAA, 1% penicillin-streptomycin, insulin, and a 1% B27 supplement (not containing vitamin A)). At 15 days after initiation of the culture, the medium was exchanged with a brain differentiation medium supplemented with a vitamin A-containing B27 supplement, and was then transferred into an Orbital Shaker. Thereafter, the medium was exchanged with a fresh one, every 4 or 5 days, until 30 days after initiation of the culture.

### 1-4. Quantitative PCR Analysis

Total RNA was extracted using TriPure Isolation Reagent kit (Roche Diagnosis). cDNA was synthesized using SuperScript (registered trademark) IV Reverse Transcriptase (Invitrogen). Quantitative PCR was carried out using KAPA SYBR Fast qPCR kit (KAPA Biosystems). The data were standardized with respect to the expression level of GAPDH.

### 1-5. Immunohistostaining

The organoid was fixed in a 4% paraformaldehyde/PBS solution at 4° C. for 1 hour, and was then washed in PBS. After that, the resulting organoid was subjected to an antifreeze treatment in 30% sucrose/PBS at 4° C. overnight. Thereafter, the organoid was embedded in Tissue-Tek O.C.T. Compound, and was then preserved at −80° C.

Thereafter, the organoid was cut into a frozen section with a size of 12 and was recovered on a slide glass. The slide glass was dried, and was then preserved at −20° C. The slide glass was washed with PBS three times, and was then incubated with a blocking buffer (1×PBS containing 1% BSA, 5% Goat serum, and 0.3% Triton X-100) at room temperature for 1 hour. Thereafter, it was incubated with primary antibodies diluted with a blocking buffer (anti-NKX2.1 antibody, 1:1000; and anti-MASH1 antibody, 1:1000) at 4° C. overnight. After completion of the incubation with the primary antibodies, the slide glass was washed with PBS three times, and was then incubated with an Alexa Fluor 488-binding secondary antibody, followed by incubation with Hoechst33342. A fluorescent image was obtained using an inverted fluorescence microscope equipped with a CMOS camera (Zyla4.2, Andor).

### 1-6. Synthesis of caged SAG (Photoresponsive Protective Group-Binding SAG)

### Synthesis of Compound 1

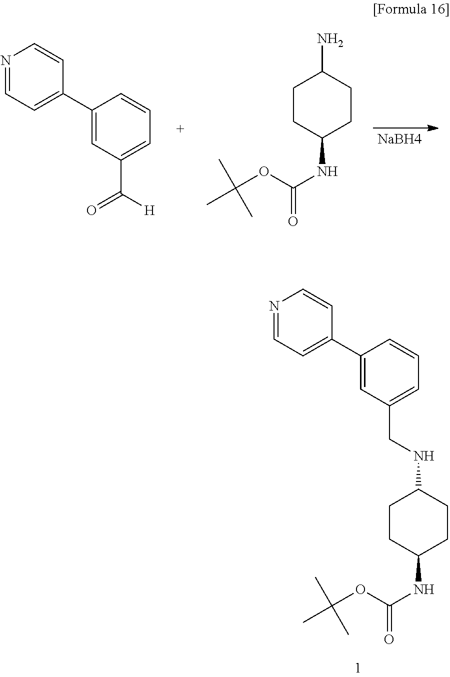

Boc-pyridine (604 mg, 2.82 mmol, 1.02 eq) was added into a pyridinylbenzaldehyde solution (504 mg, 2.75 mmol, 1 eq) dissolved in methanol, and the obtained mixture was then stirred on ice for 30 minutes. After completion of the stirring, NaBH₄ (332 mg, 8.78 mmol, 3.2 eq) was added to the reaction solution, and the obtained mixture was then stirred on ice for 20 minutes, and then at room temperature for 1 hour. A Na₂CO₃ saturated solution was added to the reaction mixture to terminate the reaction. Thereafter, the reaction mixture was extracted with chloroform (30 mL×3), and the organic layers were gathered and were then dried over Mg₂SO₄. The solvent was removed under vacuum, and the residue was then subjected to column chromatography on silica gel (DCM/MeOH=10:1) to obtain Compound 1.

Yield: 82% (857.4 mg).

[Table 6]

[symbol]

¹H-NMR (600 MHz, CDCl3): δ 1.10 -1.26 (br m, NCHCH2CH2CHN, 4H): 144 (s, (CH3)₃CO, 9H); 2.02 (t, NCHCH2CH2CHN, 4H); 2.50 (t, NCHCH2CH2CHN, 1H); 3.43 (s, CH2HCH, 1H): 3.88 (s, CH2NCH 2H); 4.37 (s, NCHCH2CH2CHN, 1H); 7.26-7.66 (m, aromatic, 5H); 8.66 (d, aromatic CH.CH═N.CH═CH, 2H).

### Synthesis of Compound 2

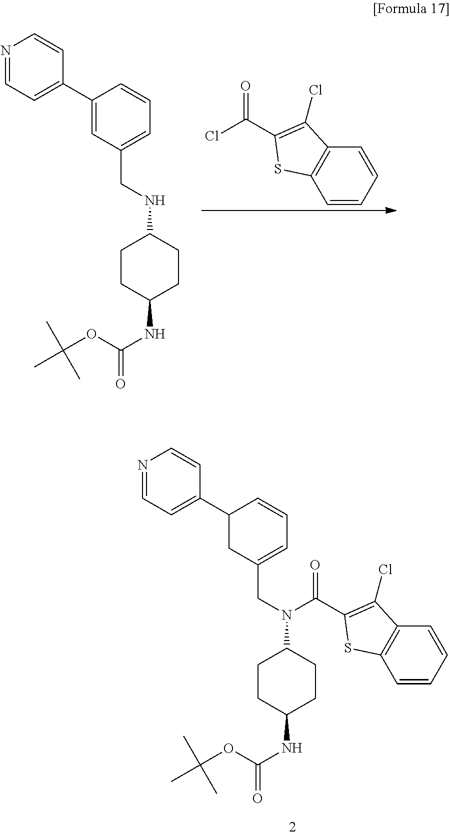

Et₃N (750 μL, 5.3 mmol, 2.4 eq) was added to Compound 1 (857.4 mg, 2.24 mmol, 1 eq) and 3-chlorobenzo[b]thiophene-2-carbonyl chloride (569.2 mg, 2.46 mmol, 1.1 eq) in a dichloromethane solution, and the obtained mixture was then stirred at room temperature for 30 minutes. The solvent and Et₃N were removed under vacuum, and the residue was then subjected to column chromatography on silica gel (acetone:hexane=1:1) to obtain Compound 2. Yield: 78% (1.00 g).

[Table 7]

[symbol]

¹H-NMR (600 MHz. CDCl3): δ 1.27 (br m, NCHCH2CH2CHN, 4H); 1.38 (s, (CH3)₃CO, 9H); 1.94 (t, NCHCH2CH2CHN, 4H); 3.30 (s, O═CNHCH, 1H): 3.79 (s, NHCHCH2CH2CHN, 1H); 4.21 (s, NHCHCH2CH2CHN, 1H); 4.83 (s, NCH2-aromatic 2H); 4.37 (s, NCHCH2CH2CHN, 1H); 7.31-7.89 (m, aromatic, 10H); 8.68 (d, aromatic CH.CH═N.CH═CH, 2H).

### Synthesis of SAG

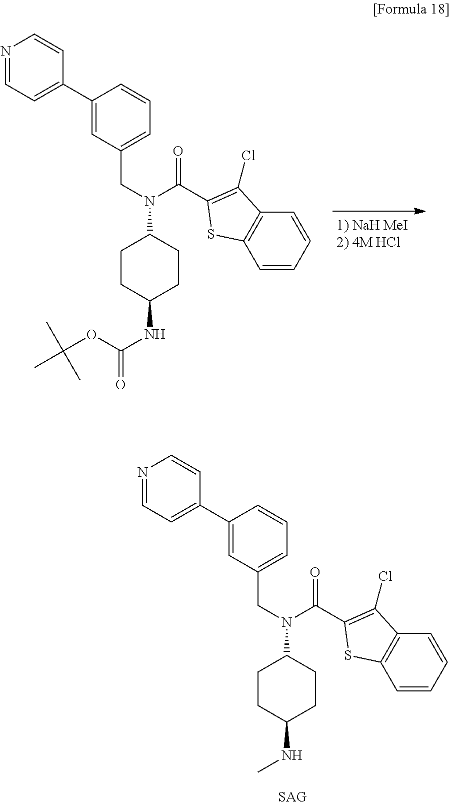

A catalytic amount of water (5 μL) was added to Compound 2 (269.6 mg, 0.468 mmol, 1 eq) in a DMF solution, and the obtained mixture was then stirred on ice for 1 hour. Subsequently, NaH (150 mg, 3.75 mmol, 8.6 eq) was added to the reaction mixture, and the obtained mixture was then stirred on ice for 1 hour. Subsequently, iodomethane (37.5 μL, 0.602 mmol, 1.3 eq) was added to the reaction mixture, and the reaction solution was then stirred at room temperature overnight. Thereafter, a Na₂CO₃ saturated solution was added to the reaction mixture to terminate the reaction. After that, the reaction mixture was extracted with dimethyl ether (60 mL×3), and the organic layers were gathered and were then dried over Mg₂SO₄. The solvent was removed under vacuum, and the residue was then added to 4 M HCl/ethyl acetate (10 mL). The reaction solution was stirred at room temperature for 30 minutes. Thereafter, the solvent was removed under vacuum, and the residue was then subjected to column chromatography on silica gel (DCM:MeOH=3:1 to DCM:MeOH=1:1) to obtain SAG. Yield: 55% (126.2 g).

[Table 8]

[symbol]

¹H-NMR (600 MHz, dDMSO): δ 1.76 (br m, NCHCH2CH2CHN, 4H); 2.98 (t, NCHCH2CH2CHN, 4H); 3.63 (s, NHCHCH2CH2CHN, 1H); 4.84 (s, NCH2-aromatic 2H); 7.53-8.21 (m, aromatic, 10H); 8.68 (d, aromatic CH.CH═N.CH═CH, 2H).

### Synthesis of Compound 3

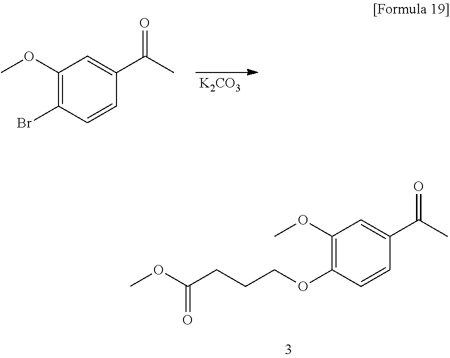

1-(4-Bromo-3-methoxyphenyl)ethanone (15.0 g, 90.3 mmol, 1 eq), K₂CO₃ (20.1 g, 144.6 mmol, 1.6 eq), and methyl 4-bromobutyrate were dissolved in DMF (100 ml), and the obtained mixture was then stirred at room temperature for 16 hours. Pure water was added to the reaction mixture until the precipitate was dissolved therein, and the reaction solution was then extracted with acetic acid and a saline solution. The organic layer was dried over MgSO₄, and ethyl acetate was then removed under vacuum. The crude product was dried under vacuum for 24 hours. A white crystal (Compound 3) was obtained from yellow oil. Yield: 93% (22.3 g).

[Table 9]

[symbol]

¹H-NMR (CDCl₃, 600 MHz) 2.20 (m, CH2CH2CH2,2H); 2.57 (t, CH2CH2H): 2.57 (s, COCH3, 3H): 3.70 (s, CH3OCO, 3H); 3.92 (s, CH3OPh, 3H); 4.15 (t, OCH2CH2, 2H); 6.89 (d, aromatic, 1H); 7.53 (s, aromatic, 1H); 7.55 (d, aromatic, 1H)

### Synthesis of Compound 4

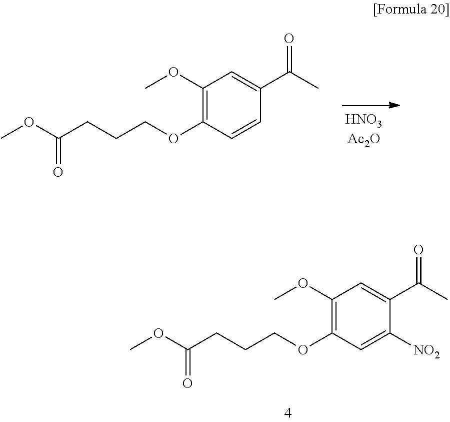

Nitric acid (200 ml, 3.3 mol) was slowly added to acetic acid (40 ml, 406 mmol) on ice. To the obtained mixture, Compound 3 dissolved in 30 ml of acetic acid was slowly added. The thus obtained mixture was stirred on ice for 2.5 hours, and thereafter, pure water (4° C.) was slowly added to the reaction mixture. The generated precipitate was recovered by filtration, and was then dried under vacuum to obtained yellow powders (Compound 4). Yield: 69% (8.02 g).

[Table 10]

[symbol]

¹H-NMR (CDCl₃, 600 MHz) 2.24 (m, CH2CH2CH2, 2H); 2.53 (s, COCH3, 3H); 2.60 (t, CH2CH2COOH, 2H); 3.74(s, CH3OCO, 3H): 3.99(s, CH3OPh, 3H); 4.19 (t, OCH2CH2, 2H); 6.78 (s, aromatic CH-m.NO2, 1H); 7.64 (s, aromatic CH-o.NO₂, 1H)

### Synthesis of Compound 5

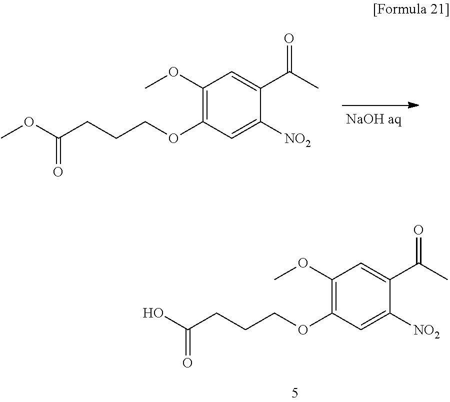 [SMILES: COC(=O)CCCOc1cc([N+](=O)[O-])c(C(C)=O)cc1OC ; COc1cc(C(C)=O)c([N+](=O)[O-])cc1OCCCC(=O)O]

1 M NaOH aq (8 mL) was added to an ethanol (40 mL) suspension of Compound 4 (1.02 g, 2.93 mmol), and the obtained mixture was then stirred at 40° C. for 1 hour. Thereafter, EtOH was removed under vacuum, and a NaHCO₃ saturated aqueous solution (20 mL) was then added to the residue, followed by extraction with DCM (20 mL×3). The organic layer was dried over MgSO₄ and by solvent evaporation, to obtain Compound 5. Yield: 87% (760 mg).

[Table 11]

[symbol]

¹H-NMR (CDCl₃, 600 MHz) 2.23 (m, CH2CH2CH2, 2H); 2.50 (s, COCH3, 3H); 2.64 (t, CH2CH2COOH, 2H); 4.00 (s, CH3OPh, 3H); 4.19 (t, OCH2CH2, 2H); 6.75(s, aromatic CH-m.NO₂, 1H); 7.63 (s, aromatic CH-o.NO₂, 1H)

### Synthesis of Compound 6

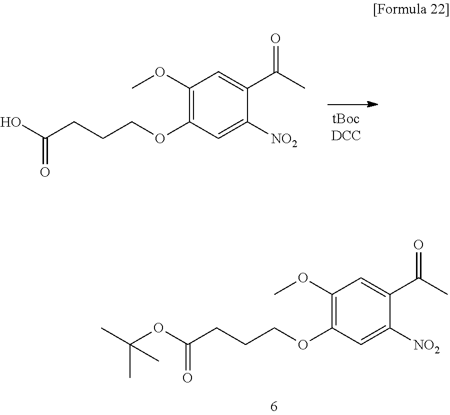

Compound 5 was dissolved in a tBuOH/THF (3:1) solution, and Boc₂O (222.6 mg, 1.68 mmol, 3 eq) in a tBuOH/THF (3:1) solution was then added to the above-obtained solution little by little over 15 minutes. The reaction solution was stirred at room temperature overnight. Thereafter, the solvent was removed under vacuum, and the residue was then subjected to column chromatography on silica gel (DCM 100%) to obtain Compound 6. Yield: 47% (92.5mg).

[Table 12]

[symbol]

¹H-NMR (CDCl₃, 600 MHz) 1.46 (s, (CH3)3C, 9H); 2.16 (m, CH2CH2CH2, 2H); 2.46 (s, COCH3, 3H): 2.50 (t, CH2CH2COOH, 2H); 3.96 (s, CH3OPh, 3H); 4.1.4 (t, OCH2CH2, 2H); 6.74 (s, aromatic CH-m.NO₂, 1H); 7.62 (s, aromatic CH-o.NO2, 1H)

### Synthesis of Compound 7

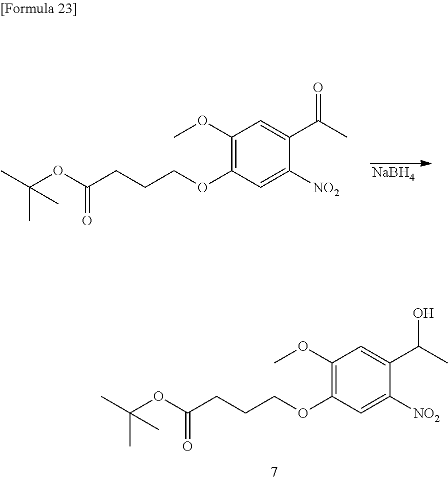

NaBH₄ (21.6 mg, 0.578 mmol, 2 eq) was added to Compound 6 (92.5 mg, 0.289 mmol, 1 e) in a methanol solution (5 mL), and the obtained mixture was then stirred on ice for 1 hour and then, at room temperature overnight. Thereafter, a citric acid aqueous solution (600 mg/mL, 10 mL) was added to the reaction solution, so that the reaction solution was converted to an acidic solution, and the solution was then extracted with chloroform (20 mL×3). The organic layer was dried over MgSO₄. The solvent was removed under vacuum, and the residue was then subjected to column chromatography on silica gel (DCM to DCM:MeOH=10:1) to obtain Compound 7. Yield: 77% (79.4 mg).

[Table 13]

[symbol]

¹H-NMR (CDCl₃, 600 MHz) 1.45 (s, (CH3)₃C, 9H); 1.56 (s, COHCH3, 3H); 2.15 (m, CH2CH2CH2, 2H); 2.45 (t, CH2CH2COOH, 2H); 3.98 (s, CH3OPh, ;3H); 4.10 (t, OCH2CH2, 2H); 5.56 (s CH3CHOH.Ph,); 7.29 (s, aromatic CH-m.NO₂, 1H); 7.57 (s, aromatic CH-o.NO2, 1H)

### Synthesis of Compound 8

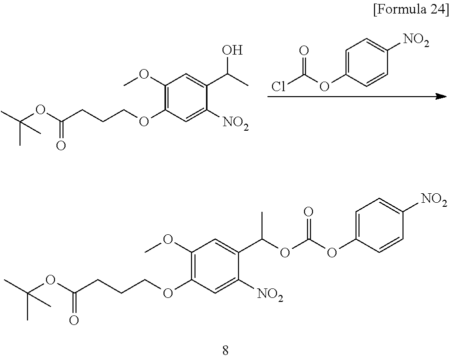

Et₃N (500 μL) was added to Compound 7 (170.0 mg, 478.3 μmol, 1 eq) and 4-nitrophenyl chloroformate (210.2 mg, 1.04 mmol, 2 eq) in a DCM solution (7 mL), and the obtained mixture was then stirred at room temperature overnight. Thereafter, the solvent was removed under vacuum, and the residue was then subjected to column chromatography on silica gel (DCM to DCM:MeOH=10:1 to DCM:MeOH=3:1) to obtain Compound 8. Yield: 82% (183.8 mg).

[Table 14]

[symbol]

¹H-NMR (CDCl₃, 600 MHz) 1.45 (s, (CH3)₃C, 9H); 1.78 (s, COHCH3, 3H); 2.15 CH2CH2CH2, 2H); 2.45 (t, CH2CH2COOH, 2H); 3.98 (s, CH3OPh, 3H); 4.12 (t, OCH2CH₂, 2H); 6.53 (s CH3CHOH.Ph,); 7.11 (s, aromatic CH-m.NO₂, 1H); 7.34 (d, aromatic CH-m.NO₂, 2H); 7.61 (s, aromatic CH-o.NO₂, 1H), 8.25 (d, aromatic CH-o.NO₂, 2H).

### Synthesis of caged SAG

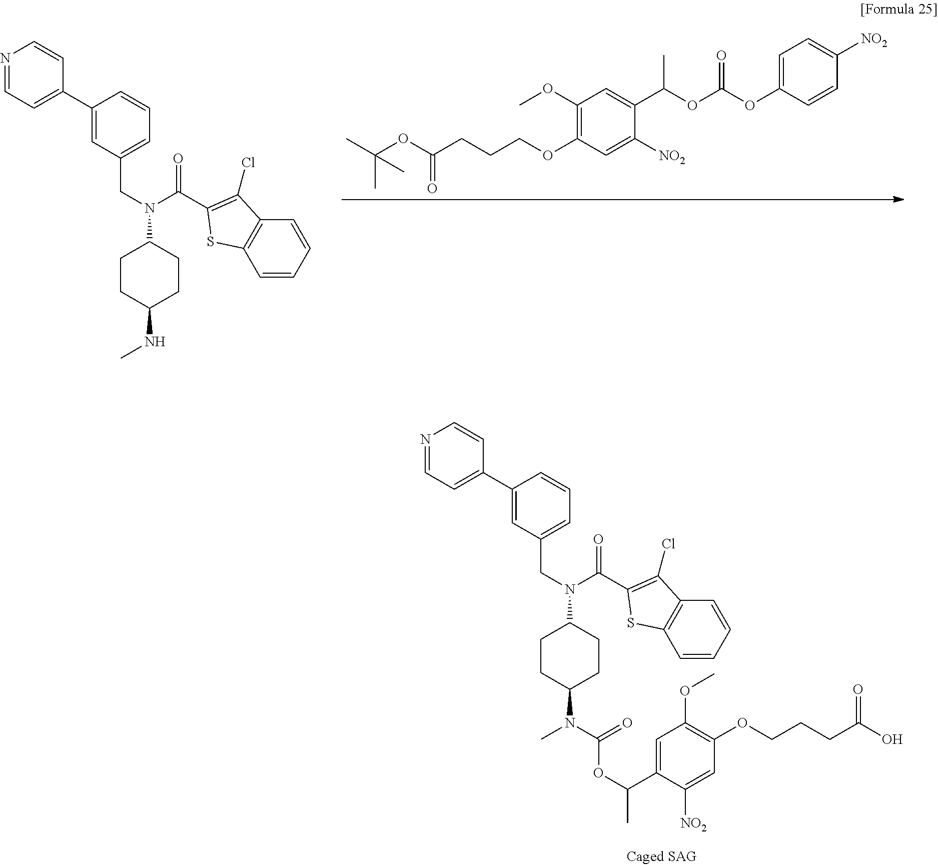

Et₃N (500 μL) was added to SAG (40.0 mg, 81.6 μmol, 1 eq) and Compound 8 (90 mg, 193.8 μmol, 2.3 eq) in a DMF solution (3 mL), and the obtained mixture was then stirred at room temperature overnight. Thereafter, the solvent was removed under vacuum, and the residue was then subjected to column chromatography on silica gel (DCM to DCM:MeOH=10:1 to DCM:MeOH=3:1) to obtain caged SAG protected by Boc groups (81.9 mg). To the residue, 5 mL of a 4 M HCl-acetic acid solution was added, and the obtained mixture was then stirred at room temperature for 40 minutes. Thereafter, the solvent was removed under vacuum, and the residue was then purified by high performance liquid chromatography (C¹⁸-AR-II column, 4 mL/min, at 0 min, CH₃CN:H₂O=50:50, at 5 min, 70:50, and at 25 min, 100:0). Yield: 45% (30.2 mg).

[Table 15]

[symbol]

¹H-NMR (CDCl₃, 600 MHz); 1.60-1.72 (br m, NCHCH2CH2CHN, 4H): 1.78 (s, CH3CHOC═O.Ph, 3H); 2.18 (m, CH2CH2CH2, 2H); 2.58 (t, CH2CH2COOH, 2H); 3.83 (s, CH3OPh, 3H); 4.12 (t, OCH2CH₂, 2H); 4.50-5.0 (s, NCH2-aromatic 2H); 6.53 (s, CH3CHOC═O.Ph, 1H); 6.83 (s, aromatic CH-m.NO₂, 1H); 7.34 (d, aromatic CH-m.NO₂, 2H); 7.43-8.90 (m, aromatic, 10H) (s, aromatic CH-o.NO₂, 1H), 8.70 (d, aromatic CH-o.NO₂, 2H).

### 1-7. Synthesis of caged SAG Derivative (Photoresponsive Protective Group-Binding Antagonist-Type SAG Derivative)

### 1-7-1. Synthesis of IR-783-SANT75

### Synthesis of SANT75

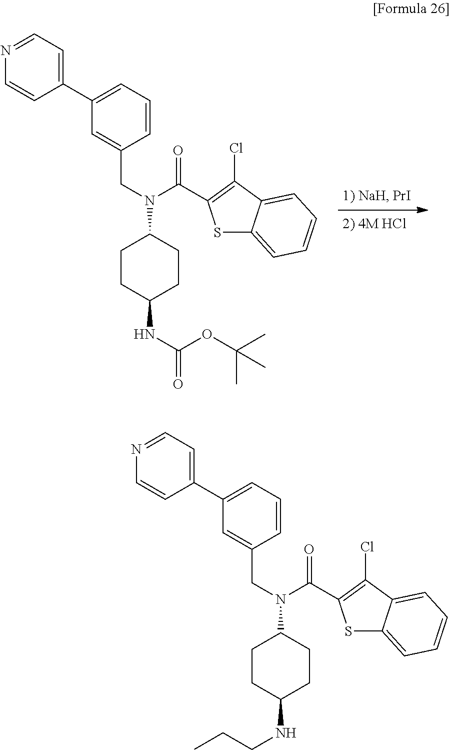

Compound 2 (Mw. 576.15, 90.1 mg, 156.4 μmol, 1 eq) was dissolved in 5 mL of dry DMF, and 5 μL of water was then added to the solution. The obtained mixture was stirred on ice for 1 hour. Thereafter, NaH (60% in paraffin oil, 60 mg, 1.5 mmol, 10 eq) was added to the reaction mixture, and the thus obtained mixture was reacted on ice for 1 hour. Thereafter, PrI (Mw. 169.9, 1.75 g/mL, 45 μM, 1.5 eq) was added to the reaction mixture, and the obtained mixture was then reacted at room temperature overnight. Thereafter, the obtained reaction mixture was extracted with 30 mL of water and 30 mL of ethyl acetate three times, and the solvent was then removed using a rotary evaporator. To the residue, 4 M HCl was added, and the obtained mixture was then reacted for 15 minutes. Thereafter, the solvent was removed using a rotary evaporator. The residue was subjected to column chromatography on silica gel (CHCl₃ to CHCl₃:MeOH=6:1 to CHCl₃:MeOH=3:1) to recover a product of interest. The amount of the product of interest was 46.5 mg, and the yield was 58%.

[Table 16]

[symbol]

¹H-NMR (600 MHz, dDMSO): δ1.23 (s, NCH2CH2CH9, 3H); δ1.42 (br, NCH2CH2CH3, 2H); δ1.75 (br m, NCHCH2CH2CHN, 4H); 1.98 (t, NCHCH2CH2CHN, 4H); 3.63 (s, NHCHCH2CH2CHN, 1H); 4.83 (s, NCH2-aromatic 2H); 7.53-8.21 (m, aromatic, 10H); 8.68 (d, aromatic CH.CH═N.CH═CH, 2H)

### Synthesis of IR-783-SANT75

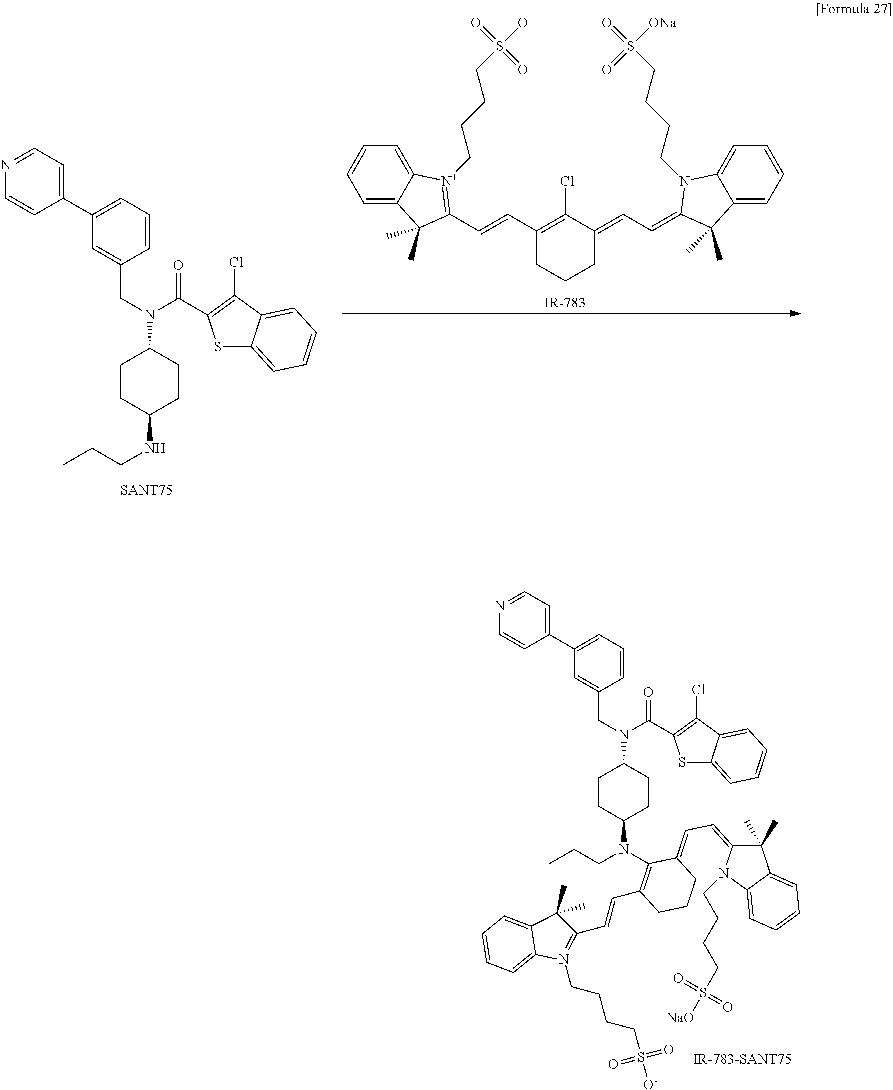

Et₃N (13 μL, 100 umol, 5.2 eq) was added to SANT75 (9.9 mg, 19.1 μmol, 1 eq) and IR-783 (21.4 mg, 28.6 μmol, 1.5 eq) in a dry DMF solution (5 mL), and the obtained mixture was then stirred at 80° C. for 1 hour. Thereafter, 30 mL of water was added to the reaction mixture, and the obtained mixture was then extracted with 15 mL of ethyl acetate three times. After that, the solvent was removed using a rotary evaporator. The residue was subjected to column chromatography on silica gel (CHCl₃ to CHCl₃:MeOH=10:1 to CHCl₃:MeOH=3:1) to recover a blue fraction. Thereafter, the solvent was removed using a rotary evaporator. Yield: 5% (0.9 mg).

### 1-7-2. Synthesis of NVOC-SANT75

### Synthesis of NVOC-SANT75

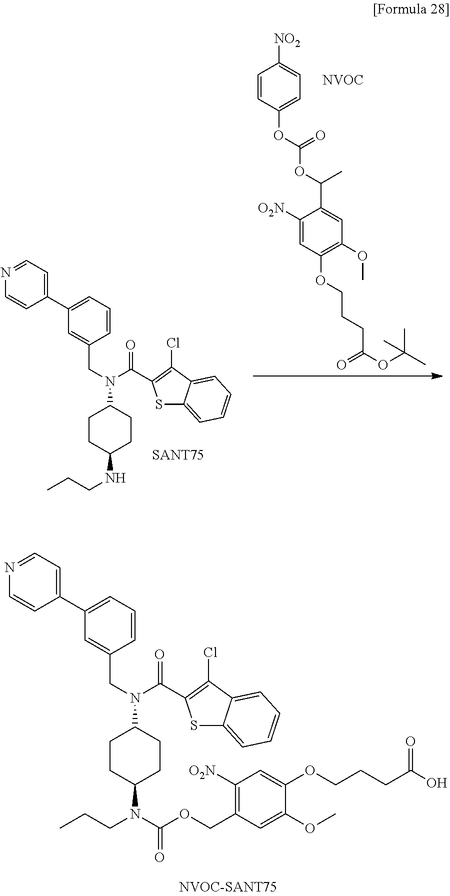

Et₃N (100 μL) was added to SANT75 (10 mg, 19.2 μmol, 1 eq) and Compound 8 (14.7 mg, 28.2 μmol, 1.4 eq) in a DMF solution (5 mL), and the obtained mixture was then stirred at room temperature overnight. Thereafter, the solvent was removed under vacuum, and the residue was then dissolved in 4 M HCl/Dioxane (1 mL) again. The obtained solution was stirred at room temperature for 30 minutes. The solvent was removed under vacuum, and the residue was then purified by column chromatography on silica gel (DCM to DCM:MeOH=50:1 to DCM:MeOH=10:1). Yield: 14% (2.34 mg).

### 1-8. Photolysis Kinetic Analysis Using HPLC

100 μM Caged SAG (PBS:DMSO=9:1) was irradiated with a light at a wavelength of 365 nm at an energy intensity of 0 to 8 J/cm². Each sample was analyzed according to HPLC (5-C₁₈-AR-II column, at 0 min, CH₃CN:H₂O=5:95, at 8 min, 30:70, and then at 15 min, 0:100). Each fraction obtained by HPLC was recovered, and was then analyzed according to ESI spectrometry.

### 2. Results

### 2-1. caged SAG

### 2-1-1. Evaluation of Properties of Caged SAG

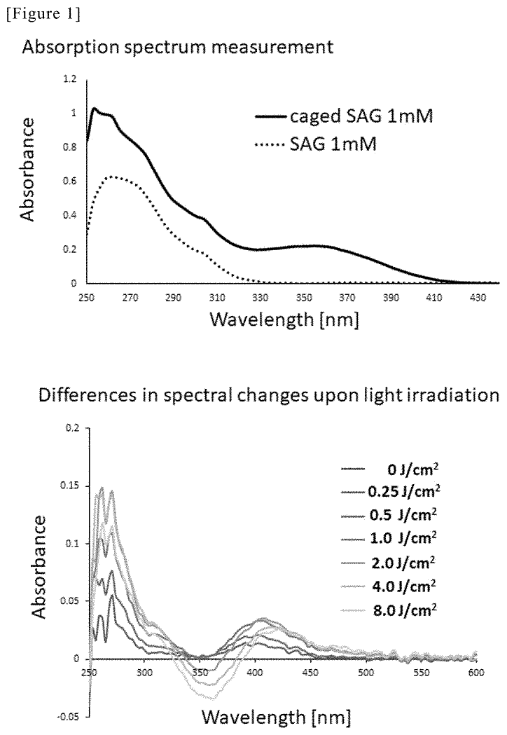

The results obtained by analyzing caged SAG, the synthetic method of which was described in the above 1-6. are shown. First, the absorption spectrum of the caged SAG was measured. SAG has a peak top around 270 nm, whereas the photodissociable protective group has a peak top around 360 nm. As a result of the measurement of the absorption spectrum, SAG had a peak only around 270 nm, whereas caged SAG exhibited a peak around 360 nm, as well as a peak at 270 nm (FIG. 1, upper view).

Subsequently, caged SAG was irradiated with a light at a wavelength of 365 nm, at energy intensities of 0.25 J/cm², 0.5 J/cm², 1.0 J/cm², 2.0 J/cm², 4.0 J/cm² and 8.0 J/cm², and thereafter, the absorption spectrum was measured. As a result, it was found that, as the energy of the irradiated light was increased, the peak around 270 nm was increased, and the peak around 360 nm was decreased.

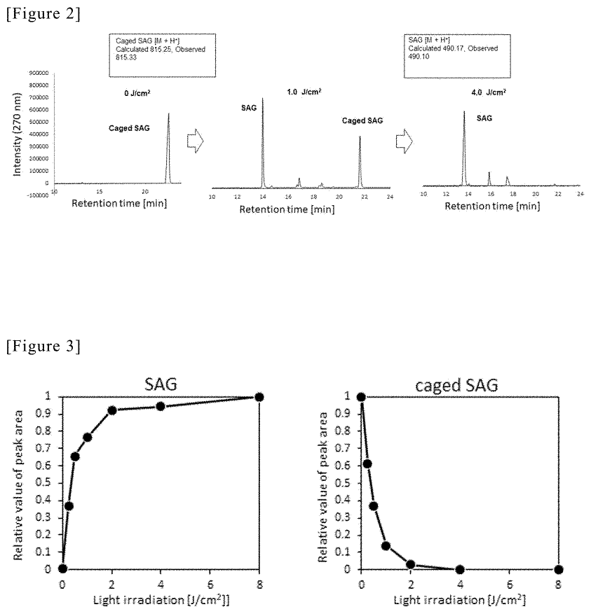

Moreover, caged SAG was irradiated with a light at a wavelength of 365 nm, at energy intensities of 0.25 J/cm², 0.5 J/cm², 1.0 J/cm², 2.0 J/cm², 4.0 J/cm² and 8.0 J/cm², and then, the caged SAG was analyzed according to HPLC. As a result, it was found that, depending on an increase in the amount of the irradiated light, the peak area of the caged SAG was decreased, and the peak area of the SAG was increased (FIG. 2 and FIG. 3). The correspondence of the peaks with the compounds was identified by the ESI Mass measurement of each fraction.

From the aforementioned results, it was confirmed that SAG was generated by irradiating the synthesized caged SAG with a light at 365 nm. In addition, it was found that photolysis was almost completed at 2-4 J/cn².

### 2-1-2. Induction of Expression of Ventral Cerebrum Markers in Cerebral Organoids by Caged SAG Irradiated with Light

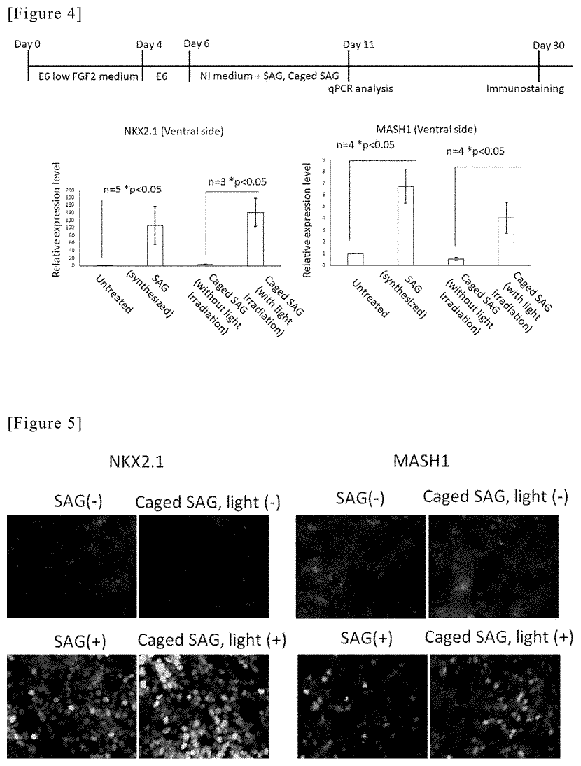

A cerebral organoid was cultured in the presence of caged SAG before light irradiation (100 nM) or caged SAG after completion of light irradiation (100 nM), and thereafter, the expression levels of the ventral cerebrum markers NKX2.1 and MASH1 were then measured according to a quantitative PCR method. The expression levels of both NKX2.1 and MASH1 were increased in the presence of the caged SAG after completion of light irradiation. On the other hand, the expression of the ventral cerebrum markers was not induced in the presence of the caged SAG before light irradiation, as in the case of the absence of SAG (FIG. 4).

From the aforementioned results, it was confirmed that the activity of caged SAG is suppressed by a photodissociable protective group, and that the caged SAG exhibits the same level of Smo agonistic activity as that of SAG as a result of light irradiation.

Moreover, the expression status of the ventral cerebrum markers (NKX2.1 and MASH1) in a cerebral organoid that had been cultured in the presence of caged SAG (100 nM) before light irradiation or after completion of light irradiation was examined according to immunohistostaining using an anti-NKX2.1 antibody or an anti-MASH1 antibody.

As a result, it was confirmed that the expression levels of NKX2.1 and MASH1 were increased when the cerebral organoid had been cultured in the presence of SAG (SAG(+)) and in the presence of caged SAG after completion of light irradiation (caged SAG(+)) (FIG. 5).

### 2-1-3. Induction of Hedgehog Signals by Light Irradiation to caged SAG

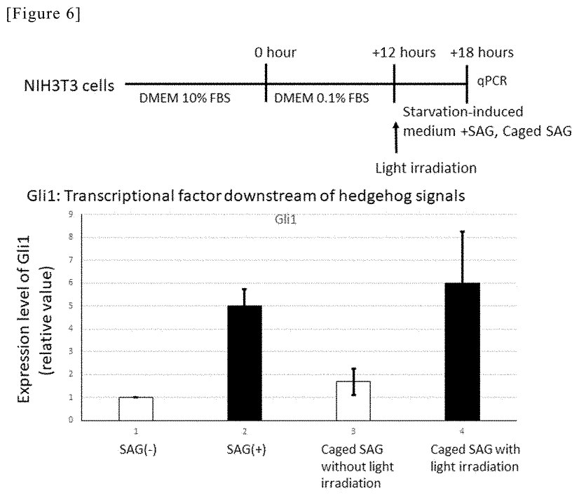

A light was applied to NIH-3T3 cells that had been cultured in the presence of caged SAG (100 nM), and the expression level of the hedgehog signal downstream gene Gli was then measured according to a quantitative PCR method. As a result, it was confirmed that, when the cells cultured in the presence of caged SAG were irradiated with a light, the expression level of Gli was increased, as in the case of culturing the cells in the presence of SAG. On the other hand, when the cells cultured in the presence of caged SAG were not irradiated with a light, an increase in the expression of Gli could not be confirmed (FIG. 6).

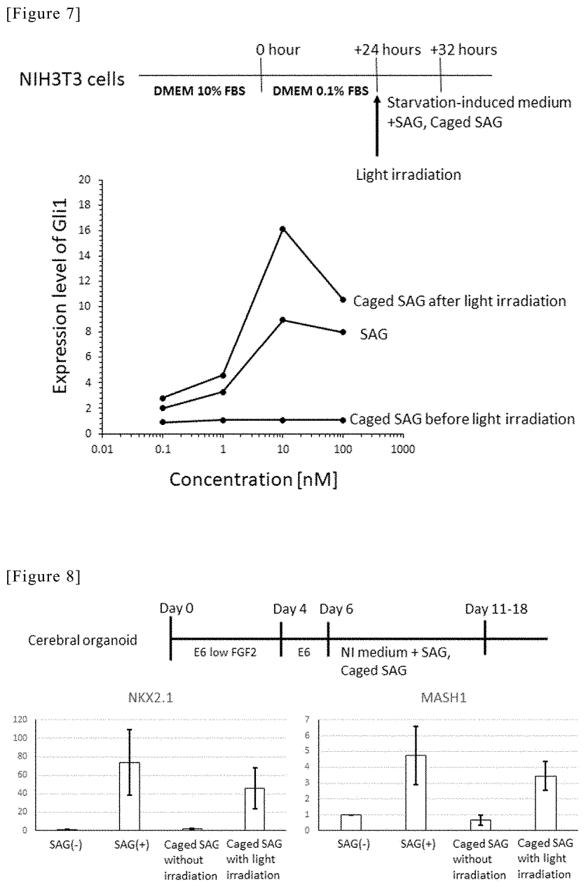

Besides, the relationship between the amount of SAG and the expression level of Gli was examined. When SAG and caged SAG (with light irradiation) were each applied at a concentration of lower than 100 nM, activation was observed at a concentration of 10 nM, but only a little activation was confirmed at a concentration of 1 nM (FIG. 7). These results are consistent with the previously reported SAG with EC50 of 3 nM (Chen et al., Proc. Natl. Acad Sci, 99: 14071-14076, 2002).

Furthermore, a cerebral organoid cultured in the presence of caged SAG was irradiated with a light, and the expression status of the ventral cerebrum markers (NKX2.1 and MASH1) was then examined according to immunohistostaining using an anti-NKX2.1 antibody or an anti-MASH1 antibody. As a result, it was confirmed that when the cerebral organoid cultured in the presence of caged SAG was irradiated with a light, the expression levels of NKX2.1 and MASH1 were increased, as in the case of culturing the cerebral organoid in the presence of SAG (FIG. 8).

### 2-2. caged SAG Derivative (Antagonist Type)

### 2-2-1. IR-783-SANT75

The properties of IR-783-SANT75 were analyzed. SANT75 that is a SAG derivative used herein is a compound exhibiting Smoothened antagonistic activity.

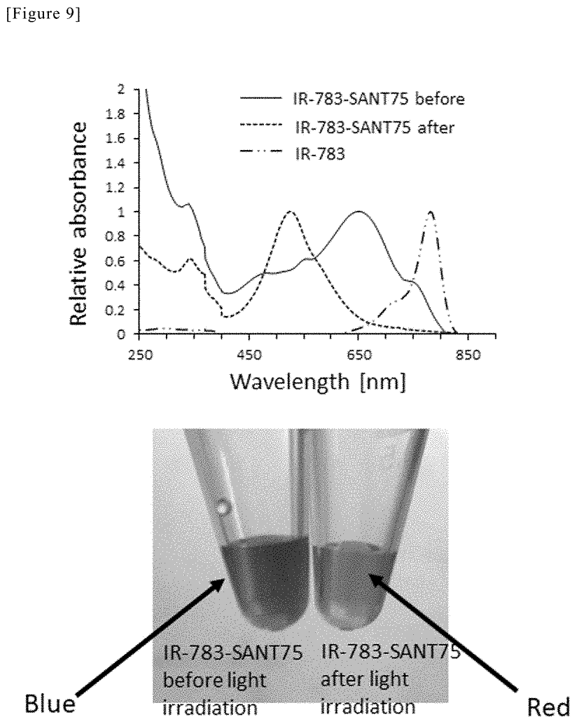

The spectrum data of IR-783-SANT75 were measured. The photodissociable protective group IR-783 is green, but if the Cl portion is replaced with N, the wavelength is shifted to a short wavelength side and the color is changed to blue. It is considered that, after irradiation with a light at 660 nm, the cyanine skeleton was oxidized, an absorber was destroyed, and the absorption wavelength was further shifted to a short wavelength side and the color was changed to red (FIG. 9). For light irradiation, a light was applied using an LED light at 660 nm for 1 hour.

### 2-2-2. NVOC-SANT75

Subsequently, NVOC-SANT75 prepared by binding the photodissociable protective group NVOC to SANT75 as an antagonist was irradiated with a light, and whether or not the antagonistic activity of SANT75 could be recovered was examined.

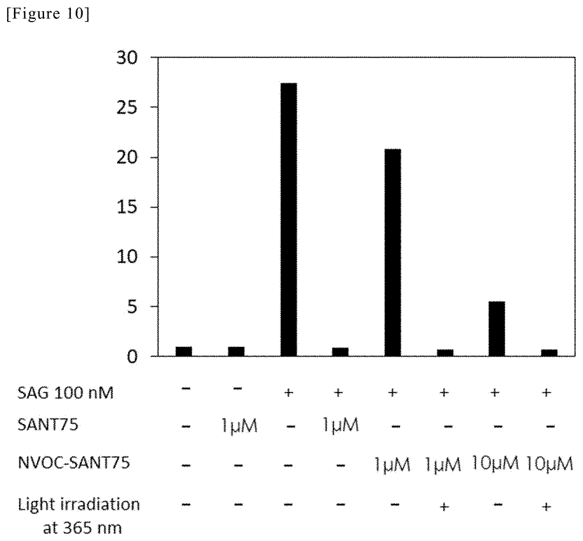

An agonist SAG (100 nM) and an antagonist SANT75 (1 μM) were allowed to simultaneously exist, and the expression level of Glil was then quantified. As a result, competitive inhibition of hedgehog signals by SANT75 was observed (FIG. 10). Further, the cerebral organoid was cultured in the simultaneous presence of SAG (100 nM) and caged SANT75 (NVOC-SANT75) (1 μM), and after completion of light irradiation, the expression level of Glil therein was then quantified. As a result, it was found that hedgehog signals were inhibited in response to light irradiation (FIG. 10).

As described above, it was confirmed that the activity of NVOC-SANT75 that is an antagonist-type caged SAG derivative is suppressed by a photodissociable protective group, and that the NVOC-SANT75 exhibits the same level of Smo antagonistic activity as that of SANT75 as a result of light irradiation.

### INDUSTRIAL APPLICABILITY

According to the present invention, it becomes possible to analyze the function of a hedgehog pathway. Therefore, it is expected to elucidate the cause of diseases associated with the hedgehog pathway and a method for treating the diseases.

1. A photoresponsive Smo ligand, in which a photodissociable protective group binds to an active site of a Smo ligand.

2. The photoresponsive Smo ligand according to claim 1, wherein the Smo ligand is Smoothened agonist (SAG) or a SAG derivative.

3. The photoresponsive Smo ligand according to claim 2, wherein the photodissociable protective group comprises an aromatic ring.

4. The photoresponsive Smo ligand according to claim 2, wherein the photodissociable protective group binds to an amino group of the SAG or the SAG derivative, to which a benzothiophene ring does not bind.

5. The photoresponsive Smo ligand according to claim 2, wherein the photoresponsive Smo ligand is represented by the following formula (II): 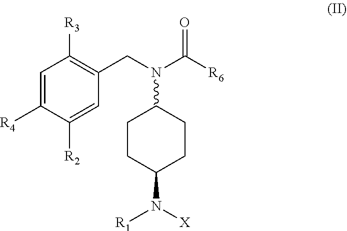
wherein X represents a photodissociable protective group; R₁ represents a hydrogen atom, or a saturated or unsaturated hydrocarbon group having 1 to 6 carbon atoms; R₂ represents hydrogen, a substituted or unsubstituted phenyl group, or a substituted or unsubstituted pyridyl group; R₃ represents hydrogen, fluorine, or a hydrocarbon group having 1 to 3 carbon atoms and optionally containing oxygen; R₄ represents hydrogen, a methoxy group, or a substituent represented by the following formula (1): 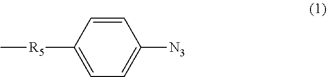
(wherein R₅ represents a hydrocarbon chain having 1 to 4 carbon atoms and optionally containing an amide bond); and R₆ represents a substituent containing a substituted or unsubstituted aromatic ring.

6. The photoresponsive Smo ligand according to claim 5, wherein the R₁ is a hydrogen atom or a methyl group.

7. The photoresponsive Smo ligand according to claim 5, wherein the R₁ is an ethyl group, a propyl group, or a 2-propenyl group.

8. A medicament or a pharmaceutical composition, comprising, as an active ingredient, the photoresponsive Smo ligand according to claim 5, a salt thereof, or a solvate or a hydrate thereof.

9. A method of inducing artificial tissues in vitro, comprising:
a step of culturing cells or tissues in the presence of the photoresponsive Smo ligand according to claim 1, and
a step of irradiating the cultured cells or tissues with a light.

10. A method of inducing artificial tissues in vitro, comprising:
a step of culturing cells or tissues in the presence of the photoresponsive Smo ligand according to claim 5, and
a step of irradiating the cultured cells or tissues with a light.

11. A method of treating developmental disorder, comprising:
administering the medicament or pharmaceutical composition according to claim 8 to a subject in need of treatment of developmental disorder.

12. A method of treating neurodegenerative disease, comprising:
administering the medicament or pharmaceutical composition according to claim 8 to a subject in need of treatment of neurodegenerative disease.

13. A method of treating bone degenerative disease, comprising:
administering the medicament or pharmaceutical composition according to claim 8 to a subject in need of treatment of bone degenerative disease.

14. A method of treating diabetes, comprising:
administering the medicament or pharmaceutical composition according to claim 8 to a subject in need of treatment of diabetes.

15. A method of treating obesity, comprising:
administering the medicament or pharmaceutical composition according to claim 8 to a subject in need of treatment of obesity.

16. A method of treating ischemia, comprising:
administering the medicament or pharmaceutical composition according to claim 8 to a subject in need of treatment of ischemia.

17. A method of treating cancer, comprising:
administering the medicament or pharmaceutical composition according to claim 8 to a subject in need of treatment of cancer.
